(19) United States
(12) Patent Application Publication
(10) Pub. No.: US 2013/0084218 A1
(43) Pub. Date: Apr. 4, 2013

(54) Anti-Bacterial Air System

(71) Applicant: Kevin Thua Ly, Pasadena, CA (US)

(72) Inventor: Kevin Thua Ly, Pasadena, CA (US)

(21) Appl. No.: 13/190,836

(22) Filed: Oct. 4, 2011

(51) Int. Cl. A61L 9/14

(52) U.S. Cl. 422/123

(57) ABSTRACT

This invention was designed to create cleaner air for healthier living. This is done with the installation of the Anti-bacterial Air System (ABAS) unit into homes or cars. This invention requires an existing A/C or car fan. The control panel of the ABAS will allow you to set the automatic timer and activate the ABAS. When the system is activated, it will mix the anti-bacterial agent and the scent, which will travel though the ¼″ PVC piping. Then the mixture will exit through the grill(s). The force and pressure of the A/C and car fan unit will facilitate the movement and spreading of the anti-bacterial and scent mist into car and room(s). Once airborne it will kill airborne bacteria and microbes and as it settles down on inanimate objects. The system will run 2-15 minutes depending on the size of home and car.

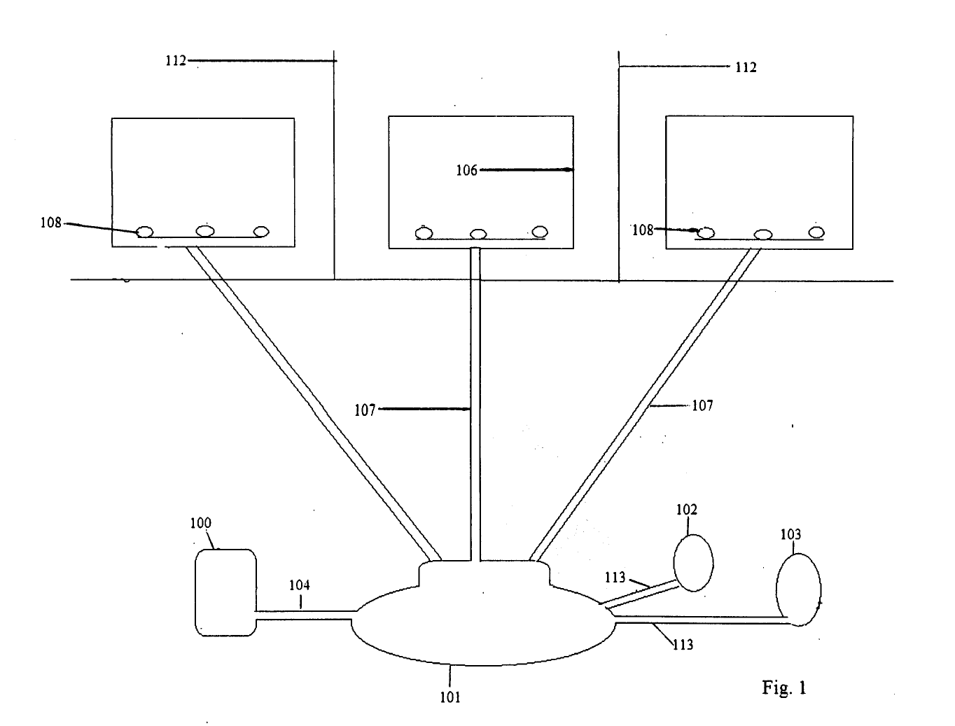

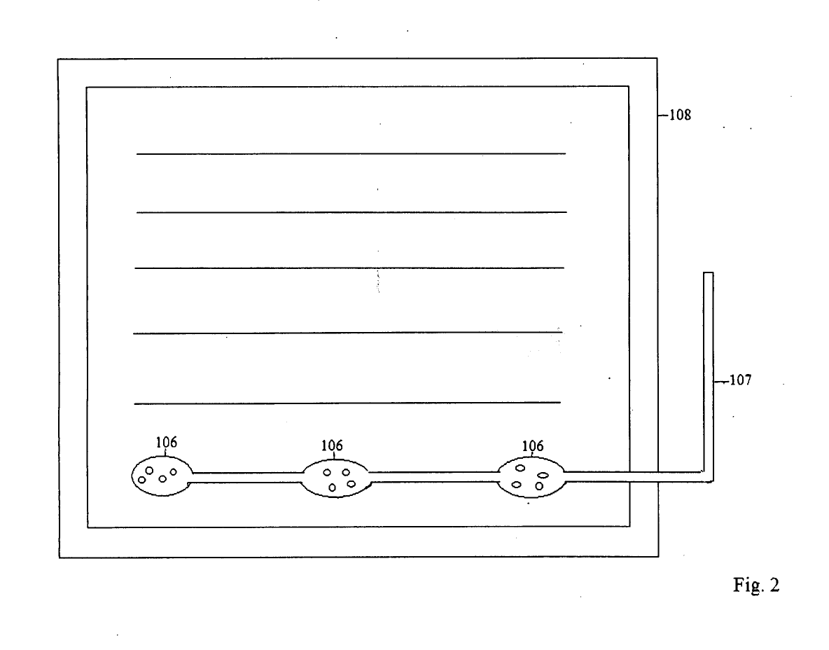

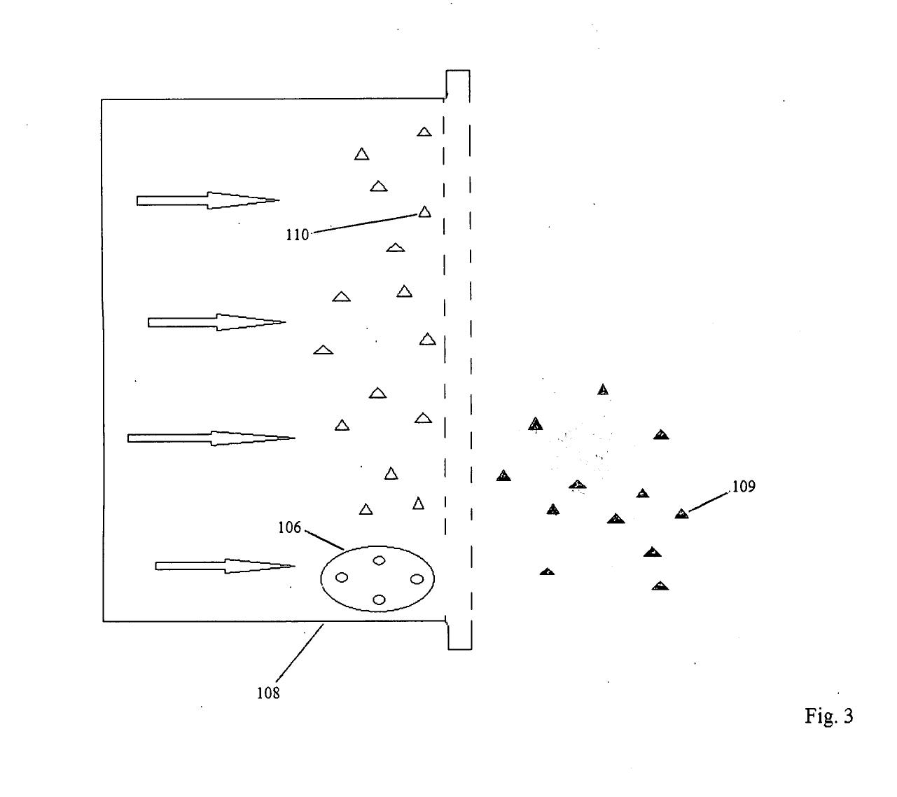

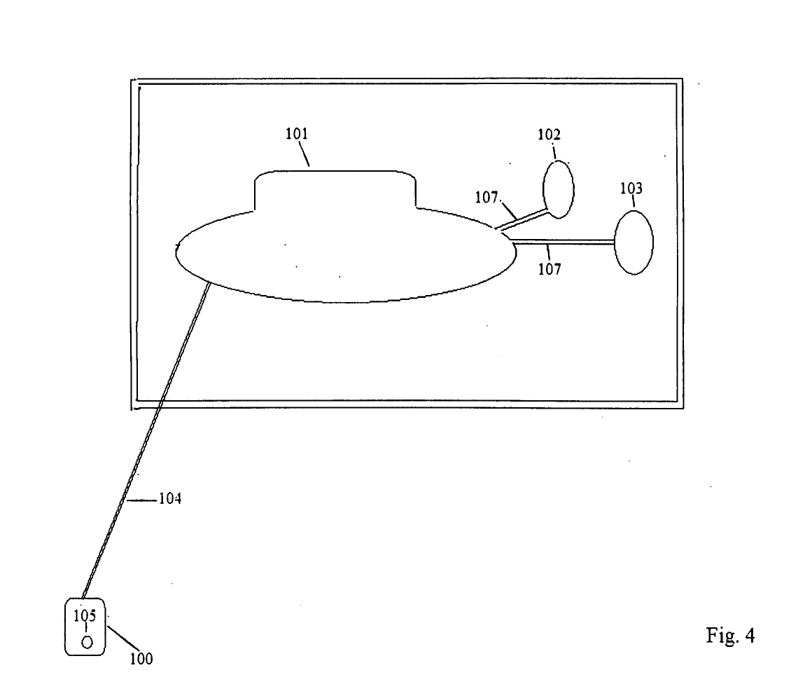

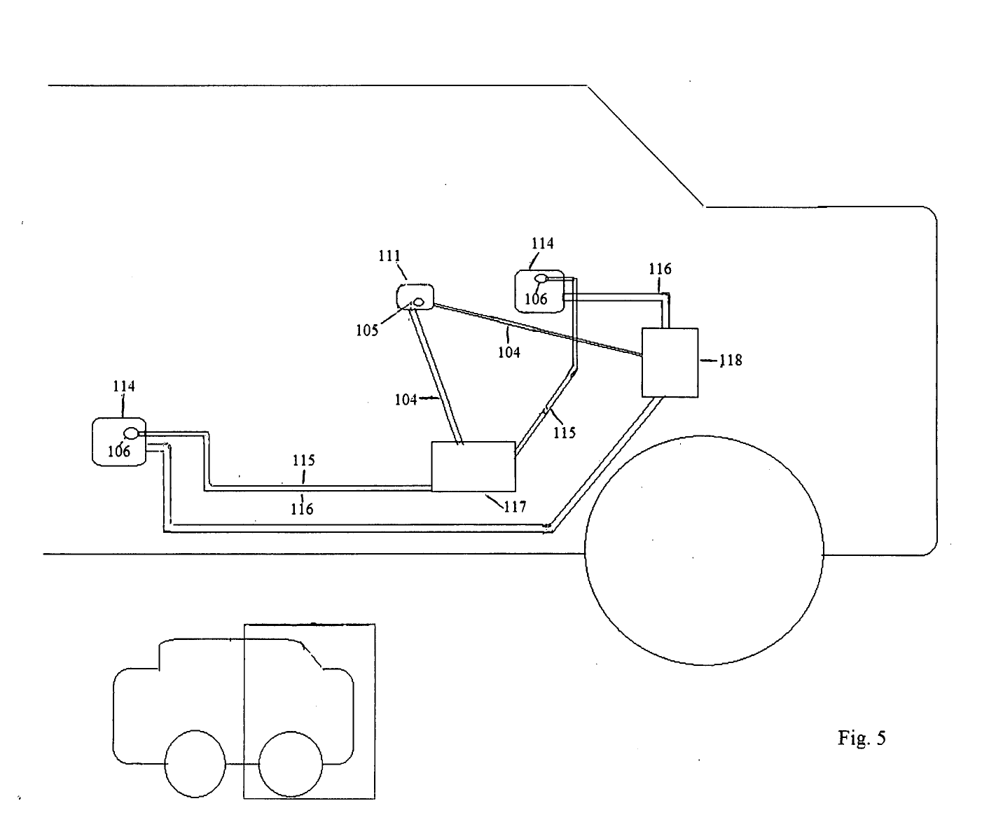

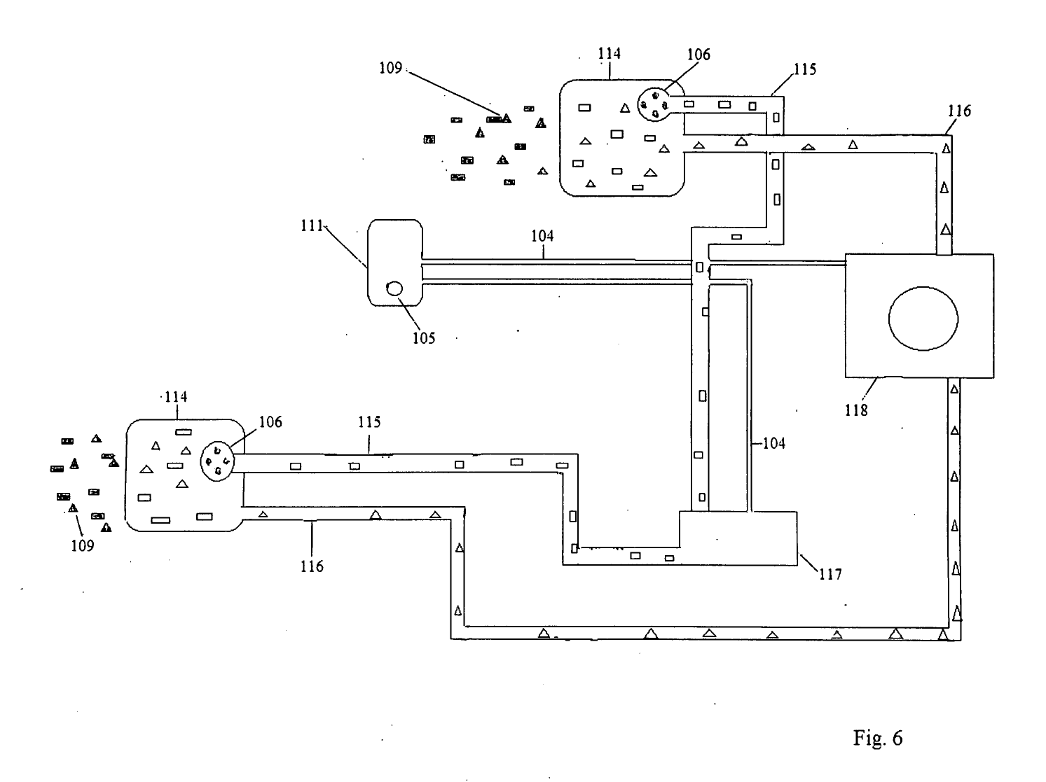

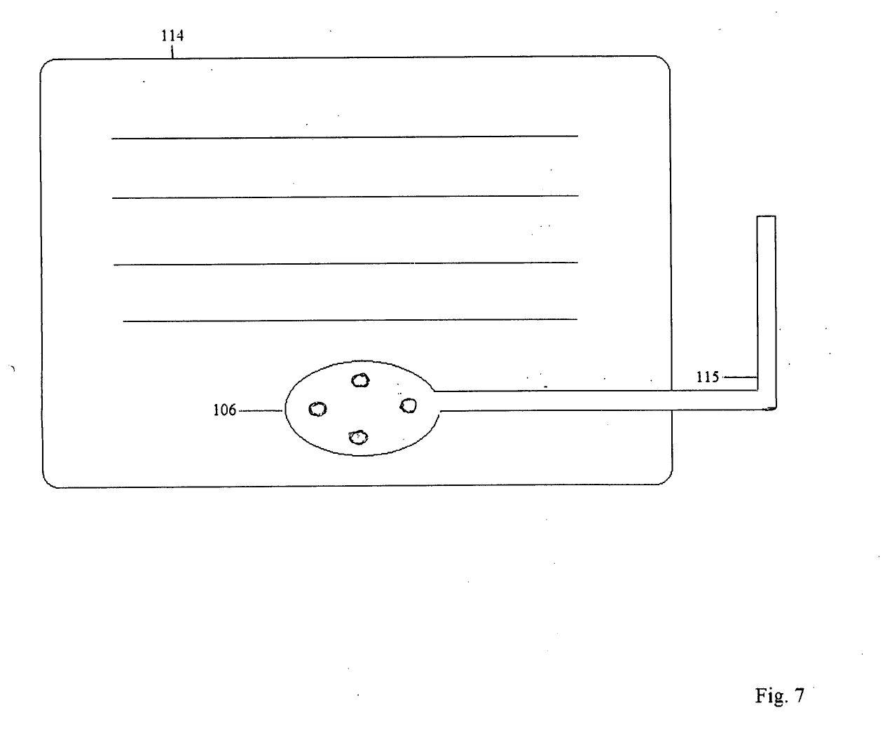

### BRIEF DESCRIPTION OF INVENTION

The Anti-bacterial Air System (ABAS) invention was created to help reduce airborne and surface bacteria in homes and cars. This invention has four parts, and the combination of these four components will create one unit (the invention). The four main components to this invention are: the anti-bacterial jar, scent jar (both of them will be made with aluminum), the compressor and the digital control timer. The jar of anti-bacterial, personalized scent jar, and control panel will be placed anywhere that is accessible in the car and house.

The fan system for the car will work to facilitate the ABAS because it will be a method of which the mixture of the anti-bacterial and scent will be pushed out of the car vent. The control panel will be place anywhere that is easily accessible in the car. The ABAS control panel will be wired up with a low voltage electrical wire to the compressor and the car fan system. This will send all necessary signals to each component of the machine. The force of the fan system will facilitate the movement of the mixture throughout the vehicle. Once it is airborne, it will kill the bacteria in the air and the bacteria on the objects inside the car, such as seat, armrest and steering wheel. ⅛″ PVC tubing lines will be run from the anti-bacterial jar and scent jar to the compressor. Then from the compressor, ⅛″ PVC will run to exit vents inside the vehicle. At the end of the ⅛″ PVC line, there will be a mist-head mounted to the car vent.

ABAS will automatically turn on once the alarm of cars is on. When the car alarm is activated, the fan will turn on, as a result, the ABAS will activate the compressor and they will all be running at the same time. The ABAS system will run for approximately two minutes, depending on the size of the vehicle. This system can also be installed inside public trains, buses and passenger airplanes.

For the home or commercial buildings, the ABAS unit will be placed in attic or basement. An A/C system is required for this invention to work. There are four components of the ABAS system, the control panel, anti-bacterial agent, personalized scent and compressor. The home A/C system and compressor will both be wired up to the control panel. Once the system is activated, it will activate the both the A/C system and the compressor at the same time. The jars of anti-bacterial, personalized scent and control panel will be placed anywhere that is accessible to the resident/owners. ¼″ PVC tubing will be run from the anti-bacterial jar and scent jar to the compressor. Then from the compressor, ¼″ PVC tubing will run to exit of the grill of the A/C system inside the house. At the end of each PVC tubing line, there will be 3 mist heads attached to the end of each line. The ¼″ PVC line will pierce through the existing A/C duct in order to meet up with the vent grills. When the compressor is activated, the anti-bacterial agent and scent will mix. Once those agents are mixed together, they will travel through the ¼″ PVC tubing and out of the vent grill. Once it leaves the vent grill, it will circulate in the room with the force and pressure of the A/C system. The A/C system will work to transport the anti-bacterial mixture because the force of the air from the A/C system will help push the mixture out of the A/C grill vents. Once it is airborne, it will kill the bacteria in the air. Once it settles down, it will kill the bacteria on the surfaces of objects inside the home. The length of time to run the A/C will depend on the size of the house, it may range anywhere from 10 to 15 minutes.

1. The Anti-bacterial Air System (ABAS) is a fast and efficient method of decrease airborne bacteria in the homes and cars. It will reduce the amount of airborne bacteria as well on bacteria on surfaces of objects.
